(19) United States
(12) Patent Application Publication
(10) Pub. No.: US 2004/0219059 A1
(43) Pub. Date: Nov. 4, 2004

(54) Heart bypass system incorporating minimized extracorporeal blood circulation system and related method of use

(75) Inventors: Carl Barringer, Smyrna, TN (US); David Fallen, Asheville, NC (US); Michael Rainone, Palestine, TX (US); Steven R. Umbach, The Woodlands, TX (US)

Correspondence Address: Hugh R. Kress, Suite 1800, 5718 Westheimer, Houston, TX, 77057, US

(73) Assignee: NovoSci Corp.

(21) Appl. No.: 10/653,522

(22) Filed: Sep. 2, 2003

Related U.S. Application Data
(60) Provisional application No. 60/467,372, filed on May 3, 2003.

(51) Int. Cl. A61M 37/00; A61M 1/14; A61M 1/34

(52) U.S. Cl. 422/44; 604/4.01

(57) ABSTRACT

A coronary bypass system incorporating a minimized extracorporeal blood circulation module is disclosed. In one embodiment, the extracorporeal blood circulation module comprises a rigid support plane for carrying the blood-handling components of the system, including an blood pump, an oxygenator, a filter, a venous blood reservoir, and a sampling manifold. The extracorporeal blood circulation module is pre-configured to interconnect all of the blood-handling modules, such that total interconnective tubing length is minimized and interfacing with an overall heart-lung bypass console can be accomplished with maximum efficiency. In one embodiment, the venous blood reservoir is of the soft-shell variety mounted over a raised platform defined in the front surface of the support plane. The raised platform further defines a indention on the front surface of the support plane. A rigid plate of the venous blood reservoir cooperates with the front surface of the support plane over the indentation to define a vacuum chamber surrounding a flexible membrane of the reservoir. A vacuum port extending into the vacuum chamber defined by the support plane and the reservoir plate is adapted to be coupled to a vacuum source, such that a regulated negative pressure can applied to the flexible reservoir membrane, thereby allowing for vacuum-assisted venous drainage.

### PRIORITY DATA

Pursuant to 35 U.S.C. § 119, this application claims the priority of prior provisional U.S. patent application Ser. No. 60467,372 filed on May 3, 2003 which provisional application is hereby incorporated by reference herein in its entirety.

### FIELD OF THE INVENTION

The present invention relates generally to medical equipment, and more particularly to an extracorporeal blood circulation system for use in cardiac bypass surgery.

### BACKGROUND OF THE INVENTION

Those of ordinary skill in the medical arts will be aware that extracorporeal blood circulation systems are utilized during cardiac surgeries to artificially oxygenate and pump blood through a patient's circulatory system. Using such systems, venous blood is diverted from entering the right chambers of the patient's heart and is instead directed to an external perfusion circuit of blood-handling components including, in most instances, an oxygenator, a filter, and a heat exchanger, before being reintroduced into the patient's circulatory system through the aorta. Due to their functionality, extracorporeal blood circulation systems are sometimes aptly referred to as “heart/lung machines.” Extracorporeal blood circulation systems have been in use for decades, and the prior art is replete with examples of such systems and their various components. Examples of extracorporeal blood circulation systems and various components thereof of particular relevance to the present invention include U.S. Pat. No. 6,337,049 to Tamari, entitled “Soft Shell Venous Reservoir,” U.S. Pat. No. 6,306,346 to Lindsay, entitled “Self-Contained Pack Assembly for an Extracorporeal Blood Circuit,” and U.S. Pat. No. 6,468,473 to Lindsay, entitled “Self-Contained Pack Assembly for an Extracorporeal Blood Circuit.” The aforementioned '049, '346, and '473 patents are hereby incorporated by reference herein in their respective entireties.

One patent application of relevance to the subject matter of the present application is U.S. patent application Ser. No. 09/390,381, filed on Sep. 3, 1999 in the name of David M. Fallen et al. and entitled “Support Devices for Surgical Systems.” The Fallen et al. '381 application is hereby incorporated by reference herein in its entirety.

Preparation for a surgical procedure involving extracorporeal blood circulation can be a complex process involving the proper interconnection of the various blood-handing components with sterile tubing. Those of ordinary skill in the art will appreciate that an extracorporeal blood circuit can be immensely complex, involving dozens of couplings and interconnections between the various blood handing elements. As a consequence of this complexity, the process of setting up an extracorporeal circuit is fraught with potential for human error to result in improper operation of the system. Moreover, the complexity of an extracorporeal circulation system exposes the patient's blood to a certain amount of foreign surfaces and substances, which can give rise to various inflammatory effects.

In all cases, it is imperative that an extracorporeal blood circulation system be designed to ensure that the blood is properly treated and handled. For instance, it is critical that the system be designed to ensure that no air bubbles are introduced into the circuit. Precautions against aeration often involve blood-handling components such as filters, defoamers and debubblers. Additionally, it is desirable to ensure that the extracorporeal blood be exposed to foreign surfaces and material to a minimum extent, as any interaction of blood with foreign substances and surfaces is likely to have inflammatory effects.

One undesirable feature of many conventional extracorporeal circulation systems is the long lengths of tubing that may be required to achieve the proper interconnection of the various blood-handling components and for interfacing the system with the patient's circulatory system. Long lengths of tubing are prone to tangling and crimping; moreover, longer overall blood circuits require greater volumes of blood to be removed from the patient, and require greater periods of time for oxygenated blood to be returned to the patient. Long lengths of interconnective tubing further undesirably increase the amount of biocompatible fluid, such as blood or saline, that must be flushed through the system upon commencement of a bypass procedure and connection of the system to a patient, sometimes referred to as the “priming volume.” The greater the priming volume, the greater the dilution of the patient's own blood, which undesirably risky and potentially harmful.

The long lengths of tubing in typical prior art bypass systems also undesirably increase the extent of exposure of the blood to foreign surfaces and materials. As would be appreciated by those of ordinary skill in the art, contact between blood and foreign surfaces and substances, including air, can undesirably have inflammatory effects on the patient's blood.

As a practical consideration, it is also important to consider the possibility that one or more blood-handling components of bypass system might fail during a surgical procedure, in the prior art, such a situation necessitated the complex process of exchanging the failed component for a working replacement. The complexity of the interconnections between the various blood handling components noted above makes such a process cumbersome and possibly dangerous, and even further increases the potential for error on the part of the perfusionist.

Another important consideration associated with coronary surgery involving extracorporeal perfusion is the amount of hemodilution, that is, the amount of fluids other than blood that must be infused into the patient's circulatory system. The need to “prime” a conventional perfusion system as it is introduced into a patient's circulatory system is one critical factor relating to the overall perfusion process.

### SUMMARY OF THE INVENTION

In view of the foregoing and other considerations, the present invention is directed to a bypass system incorporating an integrated, minimal extracorporeal circulation module.

In one embodiment of the invention, the system comprises an extracorporeal blood circulation module comprising a support plane for carrying all of the primary blood-handling elements of the bypass system, including a centrifugal pump head, a blood oxygenator, blood filter, and a venous blood reservoir. The blood handling components are rigidly and permanently affixed to the support plane in a configuration that minimizes the overall length of interconnective tubing, which is pre-configured so as to minimize the complexity of connecting the circuit to a heart-lung console.

The support plane has a substantially hollow rectangular configuration with front, back, and side surfaces. The back and side surfaces are generally planar, while the front surface is formed to define a plurality of indented and protruding supporting structures upon or within which the various blood-handling modules are mounted. The back and/or bottom surfaces is/are formed to provide for reception of mounting brackets for mounting the extracorporeal blood circulation module on a surgical support pole.

In another embodiment of the invention, the front surface of the support plane is configured to define an indentation and support frame over which a softshell venous reservoir is mounted. When the venous reservoir is mounted on the support plane, it cooperates with the support plane to define a sealed vacuum chamber between the support plane front surface and the flexible membrane of the reservoir. A vacuum fitting facilitates the introduction of a negative pressure into the vacuum chamber, such that the flexible membrane of the reservoir can be controllably drawn out, enabling vacuum-assisted venous drainage during a bypass procedure.

### BRIEF DESCRIPTION OF THE DRAWINGS

The foregoing and other features and aspects of the subject invention will be best understood with reference to a detailed description of specific embodiments of the invention, which follow, when read in conjunction with the accompanying drawings, wherein:

FIG. 1 is an illustration of a heart bypass system in accordance with one embodiment of the invention;

FIG. 2 is a front view of a minimal, integrated extracorporeal circulation module from the bypass system of FIG. 1;

FIG. 3 is a rear view of the extracorporeal circulation module from FIG. 2;

FIG. 4 is a side view of the extracorporeal circulation module from FIG. 2;

FIG. 5 is a side, cross-sectional view of the extracorporeal circulation module from FIG. 2;

FIG. 6 is a schematic diagram showing the tubing interconnections between blood handling elements in the extracorporeal circulation module from FIG. 2;

FIG. 7 is an illustration of a heart bypass system in accordance with an alternative embodiment of the invention;

FIG. 8 is a front view of a minimal, integrated extracorporeal circulation module from the bypass system of FIG. 7;

FIG. 9 is a side view of the extracorporeal circulation module from FIG. 8;

FIG. 10 is a side, cross-sectional view of the extracorporeal circulation module from FIG. 8;

FIG. 11 is a side, cross-sectional view of a venous reservoir in the extracorporeal circulation module from FIG. 2;

FIG. 12 is a side, cross-sectional view of the venous reservoir from FIG. 11, partially filled with fluid;

FIG. 13 is a side, cross-sectional view of a venous reservoir in the extracorporeal circulation module from FIG. 8; and

FIG. 14 is a side, cross-sectional view of the venous reservoir from FIG. 11, partially filled with fluid.

### DETAILED DESCRIPTION OF SPECIFIC EMBODIMENTS OF THE INVENTION

The disclosure that follows, in the interest of clarity, does not describe all features of actual implementations. It will be appreciated that in the development of any such actual implementation, as in any such project, numerous engineering and clinical decisions must be made to achieve the developers' specific goals and subgoals, which may vary from one implementation to another. Moreover, attention will necessarily be paid to proper engineering and clinical practices for the environment in question. It will be appreciated that such a development effort might be complex and time-consuming, but would nevertheless be a routine undertaking for those of ordinary skill in the relevant fields.

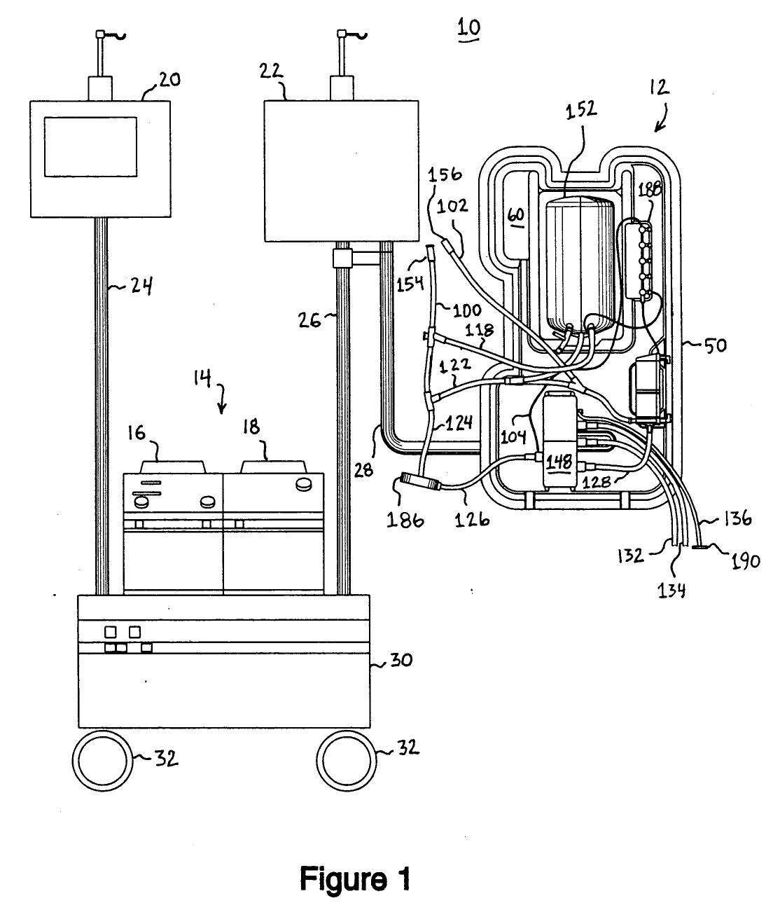

Referring to FIG. 1, there is shown a diagram of a cardiac bypass/perfusion system 10 incorporating a minimized extracorporeal blood circulation circuit 12 in accordance with the presently preferred embodiment of the invention. Bypass system 10 comprises, in addition to the extracorporeal blood circulation module 12, a heart-lung machine 14 including, in an exemplary embodiment, at least one suction pump module 16 and a cardioplegia pump module 18. In the present embodiment, heart-lung machine 14 is a Model HL-20 Perfusion System commercially available from Jostra Corp., The Woodlands, Texas, although those of ordinary skill in the art will recognize that the present invention is not limited to practice with this particular heart-lung machine.

Associated with heart-lung machine 14 is a display panel 20 for displaying such information as arterial pressure, ECG, pulsatile flow, and the like, in accordance with conventional practice. Also associated with heart-lung machine 14 is a control panel 22 for enabling a perfusionist to control overall operation of the system, again in accordance with conventional practice in the art.

The chassis 30 of heart-lung machine 14 carries a plurality of support poles 24, 26, and 28 for supporting the display panel 20, control panel 22, and extracorporeal blood circulation module 12. Wheels 32 are preferably provided for enabling system 10 to be conveniently located and relocated in the surgical environment.

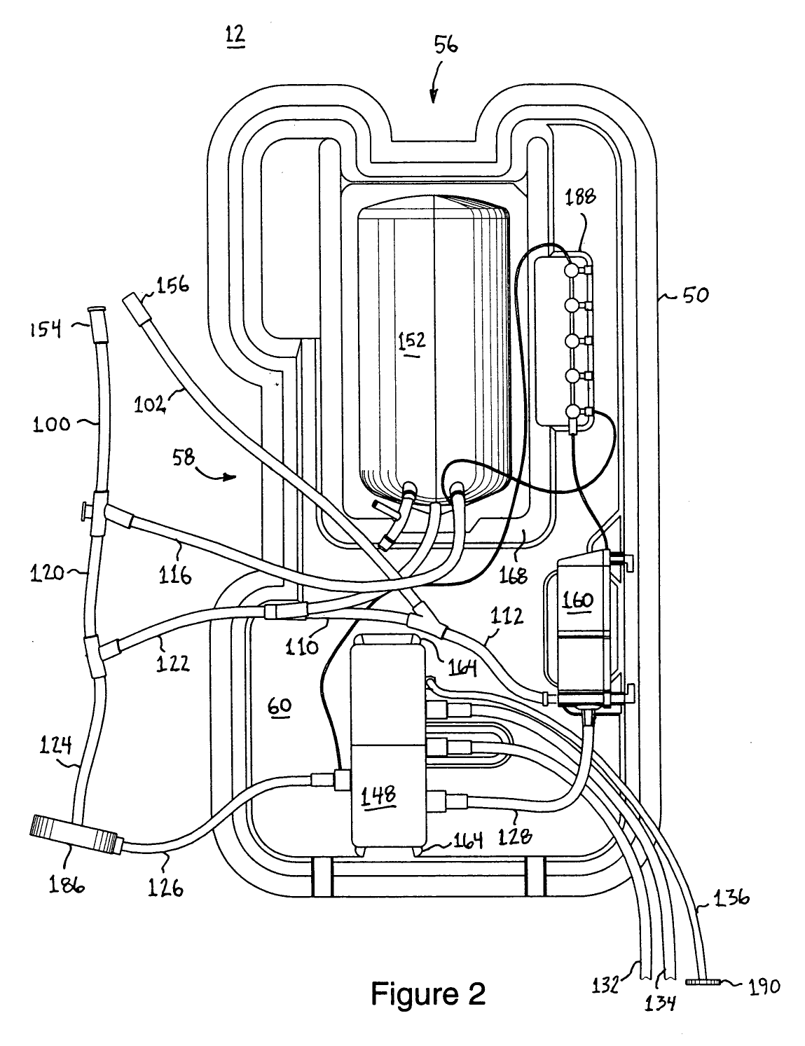

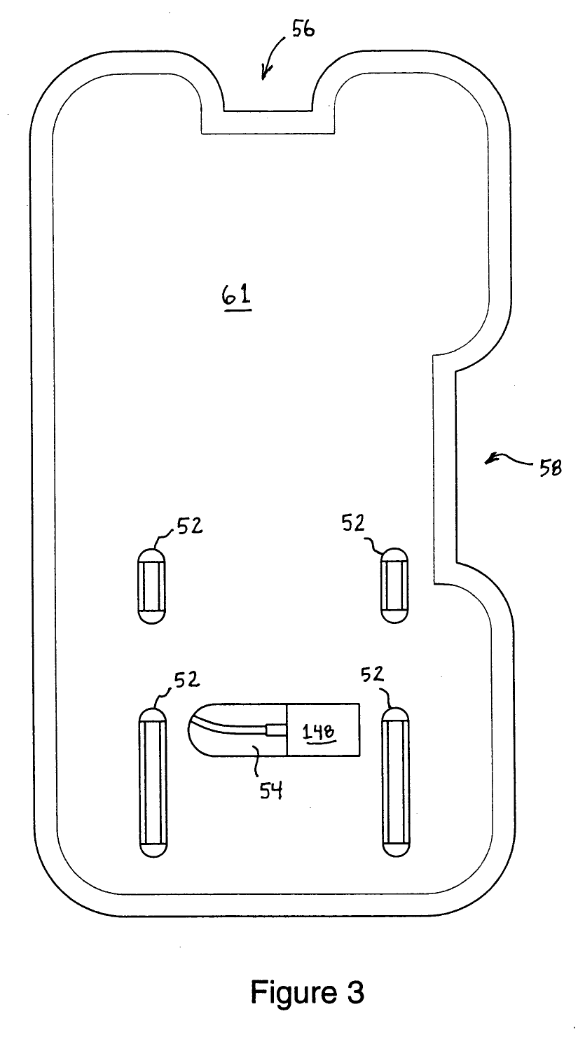

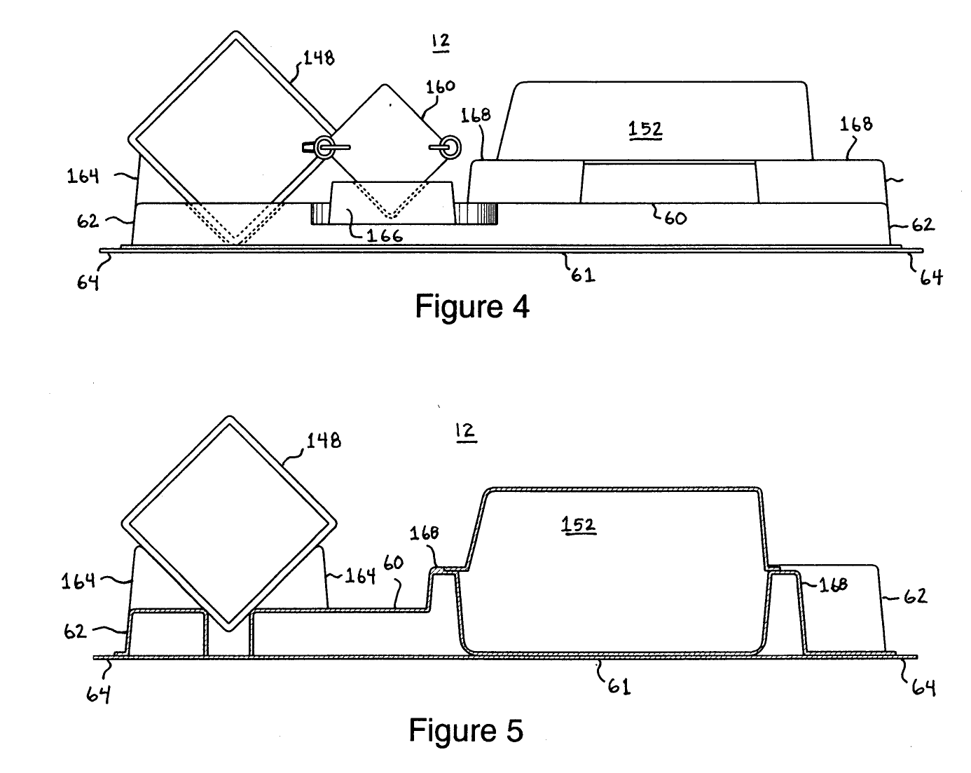

Turning to FIGS. 2, 3, 4, and 5, there are shown front, back, side, and side cross-sectional views, respectively, of extracorporeal blood circulation module 12 in accordance with one embodiment of the invention. (It is to be noted that in the interests of clarity, the interconnective tubing between the various blood-handling elements of extracorporeal circulation module 12 are omitted from FIGS. 4 and 5.)

As shall be described herein in further detail, extracorporeal blood circulation module 12 possesses a number of desirable features that lead to highly beneficial consequences both for the perfusionist and the patient undergoing cardiac bypass using system 10.

In accordance with one aspect of the invention, extracorporeal blood circulation module 12 is an integrated, self-contained module of a minimal physical size, and consists of all of the fundamental components required for extracorporeal circulation, oxygenation, filtering, temperature control, flow monitoring, and other functions. In the preferred embodiment, extracorporeal blood circulation module 12 is shipped from the manufacturer to the end-user fully populated with all of the functional components and having all tubing and other fluid paths fully connected, such that it is not necessary for the perfusionist to assemble or connect any of the functional components of the module prior to use.

The self-contained, fully integrated configuration of extracorporeal blood circulation module 12 is made possible through the provision of a support plane 50 upon which the various functional components are mounted. In the preferred embodiment, support plane 50 comprises a substantially parallelepiped-shaped, three-dimensional structure (with rounded corners) and having front, back and side surfaces. The back side 61 of the support plane is substantially planar, with apertures 52 corresponding to structures for receiving mounting bracket arms, as well as an aperture 54 for ensuring sufficient sterilization gas flow. Indentations 56 and 58 around the top and side, respectively, of support plane 50 are similarly provided to ensure adequate sterilization gas flow prior to packaging of extracorporeal blood circulation module 12 for shipment to an end-user.

In the presently preferred embodiment of the invention, support plane 50 is hollow, and has a substantially rectangular, parallelepiped configuration, except that a front surface 60 of support plane 50 has various indenting and protruding features formed therein to accommodate reception and securing of the blood-handling elements of extracorporeal blood circulation module 12, as will be hereinafter described in further detail. (Also, sides 62 of support plane 50 may be sloped inward slightly from bottom to top to accommodate reception of extracorporeal circulation module 12 into a shipping container; a perimeter flange 64 may also be provided to further accommodate securing of module 12 into such a container.) Support plane is preferably made of thermoformed high-density polystyrene having a thickness on the order of 0.070 to 0.090 inches, although other materials of similar thickness, sterilizability, rigidity, and durability, including without limitation, ABS, may be used. In one embodiment, the back 61 of support plane is formed separately from front 60 and sides 62, and the two pieces are glued or otherwise permanently bonded together.

As noted above, support plane functions to carry the blood-handling components of extracorporeal blood circulation module 12. Referring in particular to FIGS. 2 and 4, these include an oxygenator 148, a venous blood reservoir 152, and a filter 160. In the presently disclosed embodiment, oxygenator 148 is a QUADROX® Membrane Oxygenator Model HMO 1030, commercially available from Jostra Corp., The Woodlands, Tex. Filter 160 is in the presently preferred embodiment is a Quart® Arterial Filter, Model No. HBF140, commercially available from Jostra Corp. While these blood handling elements are believed to be well-suited to the practice of the present invention, those of ordinary skill in the art having the benefit of the present disclosure will appreciate that the present invention is by no means limited to practice using these specific models.

As can be observed in FIGS. 4, and 5 a pair of sloped protruding structures 164 are formed in front 60 of support plane 50, thereby defining a substantially triangular indentation into which a corner of oxygenator 148 is received. In keeping with the fully integrated configuration of the invention, oxygenator is permanently secured onto the front 60 of support plane 50, such as with glue, double-sided tape, mechanical fasteners, or the like.

Similarly, a protruding structure 166 is formed in front 60 of support plane 50 to define a substantially triangular indentation into which a corner of filter 148 is received. Filter may be permanently secured within structure 166; although in some embodiments, it may be deemed desirable for filter 148 to be releasably secured within structure 166, as some perfusionists may prefer to have the ability to visually inspect filter 148 during the course of a bypass procedure.

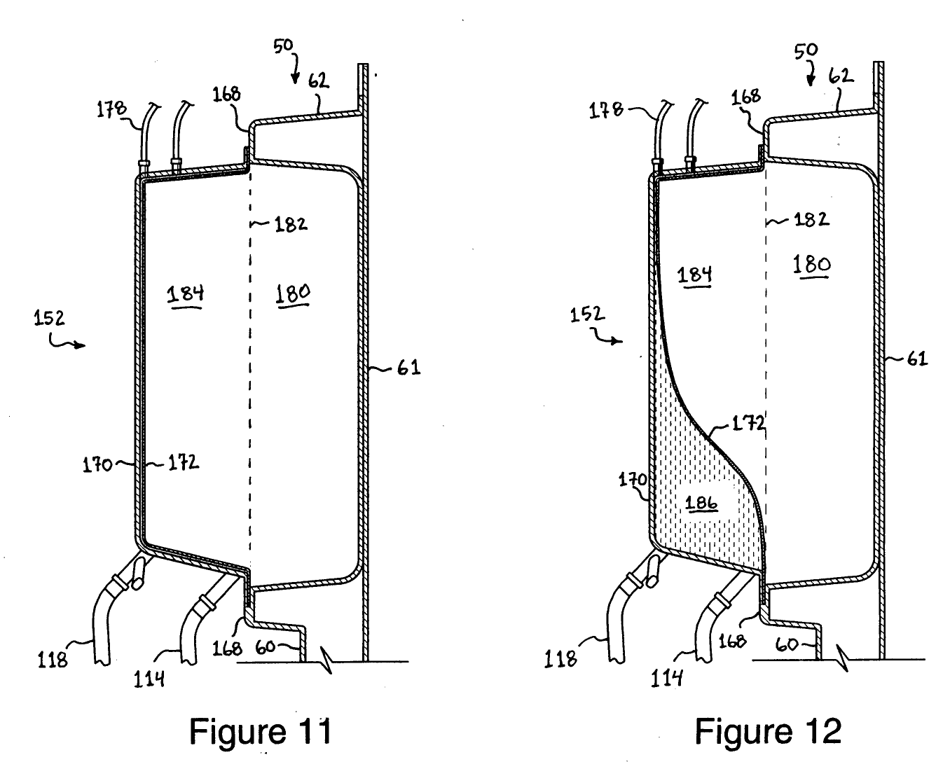

Reservoir 152 is mounted atop a hollow, raised platform 168 formed in the front 60 of support plane 50. In the presently disclosed embodiment, reservoir 152 is a so-called “soft-shell” venous reservoir such as the commercially-available William Harvey® H5441VR Softshell Venous Reservoir. Reservoir 152 is best described with reference to FIGS. 11 and 12, which shows raised platform 168 on the portion of support plane 50 to which reservoir 152 is mated. As shown in FIGS. 11 and 12, reservoir comprises a concave rigid plate 170 defining its exterior, and a flexible membrane or lining 172. Membrane 172 is bonded around the perimeter of plate 170 so as to form a vacuum-tight seal. In the presently preferred embodiment, external plate is made of BASF Terluran 2802TR ABS, and membrane 172 is made of Miles Texin 285 Resin, Polyester Urethane film, having a thickness of 0.040 inches.

Reservoir 152 is provided with a venous port 174 adapted to be coupled to a patient's venous cannulus via tubing 118 and 100 and a reservoir outlet port 176, each disposed generally at the bottom of plate 170, and a priming fluid port 178 disposed generally at the top of plate 170.

As shown in FIGS. 11 and 12, reservoir 152 is mounted atop a concave indentation 180 formed in front surface 60 of support plane 50. A dashed line 182 in FIGS. 11 and 12 delineates the border between concave indentation 180 in support plane front 60 and the concave interior 184 of reservoir 152.

As shown in the Figures, the mating of reservoir 152 with the front surface 60 of support plane 12′ results in the definition of a vacuum- and fluid-tight chamber 180/184 in fluid communication with flexible wall 172 of reservoir 152. Various methods are contemplated as being suitable for mechanically fastening reservoir 152 to the front surface 60 of support plane 12′ so as to establish a fluid- and vacuum-tight seal. In one embodiment, an annular gasket corresponding generally to the shape of raised platform 168 can be provided, and the reservoir can be secured with rivets, screws, or other suitable mechanical fasteners. Suitable gasket materials include, without limitation, Buna-N Nitrile rubber, closed-cell polyurethane foam, silicone rubber, and closed cell acrylic foam. Alternatively, an adhesive material on a suitable carrier can be provided to eliminate the needs for rivets. A polyester film or acrylic closed-cell foam carrier coated on both sides with pressure-sensitive rubber-based adhesive can be used, or adhesive without a carrier can be used.

FIG. 11 shows reservoir 152 in an unfilled state, with flexible membrane 172 substantially adjacent to the interior wall of plate 170. On the other hand, FIG. 12 shows reservoir 152 in a partially filled state, with blood and/or other fluid 186 occupying a portion of the vacuum chamber defined by the interior 184 of reservoir 152 and the depression 180 of support plane 50. In essence, membrane 172 inflates into the volume of the vacuum chamber 180/184 as blood is introduced into reservoir 152 via venous input port 118.

A further element associated with support plane 50 is a blood pump connector 186 adapted in the presently preferred embodiment to interface with a centrifugal blood pump drive (not shown in the Figures). In the preferred embodiment, the blood pump utilized with system 10 is a RotaFlow® centrifugal blood pump having a spinning rotor with flow channels that impart motion to the blood. The RotaFlow® pump is commercially available from Jostra Corp.

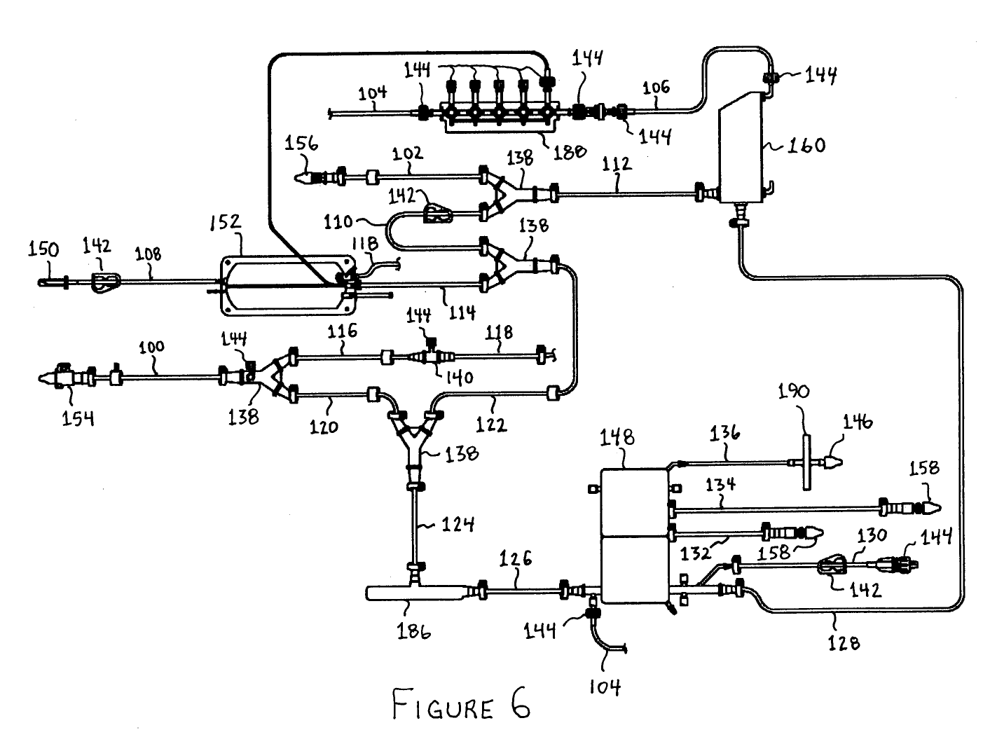

As noted above, one feature of the present invention is the minimization of distances between the various blood-handling elements of the overall system 10, which thereby reduces the overall hemodilution factor and minimizes the exposure of blood to foreign surfaces and materials. Referring to FIG. 6, there is shown a schematic diagram of the circulatory pathways of extracorporeal blood circulation module 12, showing the interconnection of the various blood-handling elements integrated into extracorporeal blood circulation module 12 (and 12′). The following Table 1 specifies various parameters of the interconnective components of extracorporeal blood circulation module 12.

TABLE 1

| REFERENCE | LENGTH | DIAMETER | TUBE THICKNESS |
|---|---|---|---|
| NO. | (inches) | (inches) | (inches) |
| 100 | 13 | 3/8 | 3/32 |
| 102 | 17 | 3/8 | 3/32 |
| 104 | 24 | 1/8 | 1/16 |
| 106 | 5 | 1/8 | 1/16 |
| 108 | 4 | 1/4 | 1/16 |
| 110 | 4 | 1/4 | 1/16 |
| 112 | 3 | 3/8 | 3/32 |
| 114 | 7 | 3/8 | 3/32 |
| 116 | 9 | 3/8 | 3/32 |
| 118 | 4 | 1/2 | 3/32 |
| 120 | 4 | 3/8 | 3/32 |
| 122 | 5 | 3/8 | 3/32 |
| 124 | 7 | 3/8 | 3/32 |
| 126 | 7 | 3/8 | 3/32 |
| 128 | 7 | 3/8 | 3/32 |
| 130 | 4 | 1/4 | 1/16 |
| 132 | 12 | 1/2 | 3/32 |
| 134 | 12 | 1/2 | 3/32 |
| 136 | 20 | 1/4 | 1/16 |

It is contemplated that with the tubing lengths substantially in accordance with Table 1 above, a total priming volume on the order of 1000-1500 cubic centimeters, and perhaps less, can be achieved.

In the presently preferred embodiment of the invention, all of the interconnective tubing in the extracorporeal circulation module 12 is made of Heparin-coated Bypass 70 Medical Grade PVC, and is commercially available from various sources, including Jostra Corp. Extracorporeal circulation module 12 also includes a plurality of “Y” connectors 138, straight connectors 140, and clamps 142. In one embodiment, “Y” connectors 138 are made of clear polycarbonate material, and selected “Y” connectors and other interconnective elements are provided with Luer connectors 144, as would be familiar to those of ordinary skill in the art. Clamps 142 are made of a suitable plastic material.

In accordance with one significant aspect of the invention, there are a minimal number of connections required to interface extracorporeal circulation module 12 with the remaining components of bypass system 10 and with a bypass patient. In particular, the primary connections consist of a connector 146 for providing oxygenating gas to oxygenator 148, a connector 150 for providing priming fluid for blood reservoir 152, a connector 154 for coupling venous line 100 to the venous catheter (not shown) that is inserted into the patient to divert blood into bypass system 10, a connector 156 for coupling arterial line 102 to an arterial cannulus inserted into the patient to provide a return path for blood from bypass system 10, and two connectors 158 for inflow and outflow connection of temperature-controlled water to oxygenator 148.

The minimal number of external connections to extracorporeal circulation module 12 is believed to be a particularly desirable feature of the present invention, inasmuch as it enables extracorporeal circulation module 12 to be readily installed as a component of bypass system 10 as an integral unit. Not only does this improve the efficiency with which bypass unit 10 can be initially set up for a bypass procedure, but in the event that a blood-handling element were to fail during a bypass procedure, extracorporeal circulation module 12 can be swiftly swapped-out for a replacement with minimal complexity.

Support plane 50 further carries a five-gang Luer-lock manifold 188 adapted to receive sampling lines from various points within the circulatory pathways of extracorporeal circulation module 12, as depicted in the Figures. Finally, support plane carries on tubing 136 a gas filter 190 at a distal end of tubing 190 for filtering oxygenating gas provided to oxygenator 148.

In operation, venous reservoir 152 functions to accommodate variations in the total volume of blood circulating extracorporeally during a bypass procedure. As would be known by those of ordinary skill in the art, at least two primary modes of operation are available with the system 10 as thus far described. During “normal” operation, tubing 118 and 122 are clamped closed, permitting venous blood from the patient to flow directly through tubing 100 and 124 to pump flow connector 186, and thence through tubing 126 to oxygenator 148, through tubing 128 to filter 160.

In the event it was desired to capture a certain volume of blood in venous reservoir 152, tubing 124 is clamped closed, and tubing 118 and 122 is unclamped, allowing gravity drainage of venous blood into reservoir 152.

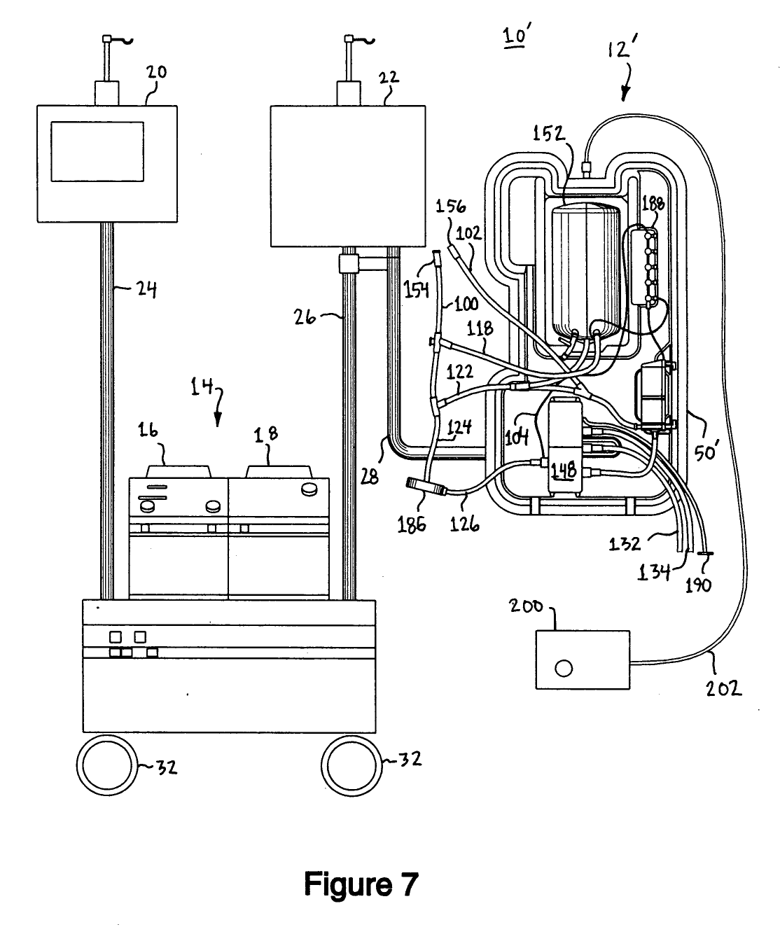

As would be appreciated by those of ordinary skill in the art, in some cases, gravity drainage of venous blood into reservoir 152 provides an inadequate rate of blood return through pump 148. Consequently, it has been proposed in the prior art to assist or augment venous drainage by applying negative pressure (suction) to the venous line. It has been alleged in the prior art that among the benefits of such so-called “vacuum assist venous drainage” or “VAVD” are the potential reduction in the inner diameter of the venous line, leading to a reduction in prime volume, as well as the potential for reduction in the size of the venous cannula. An example of a prior art VAVD reservoir is described in the above-referenced U.S. Pat. No. 6,337,049 to Tamari, entitled “Soft Shell Venous Reservoir.”Referring now to FIG. 7, there is shown a heart bypass system 10′ in accordance with an alternative embodiment of the invention, incorporating a VAVD extracorporeal blood circulation module 12′. In the description of this alternative embodiment, it is to be understood that certain components thereof that are identical to those previously described with reference to FIGS. 1-7 and 11-12 shall be identified with identical reference numerals and will not be described in any further detail.

As shown in FIG. 7, the bypass system 10′ in the alternative embodiment comprises all of the components of bypass system 10 as described with reference to FIGS. 1-6 and 11-12, and further includes a vacuum source 200 coupled to an alternative embodiment of an extracorporeal blood circulation module 12′.

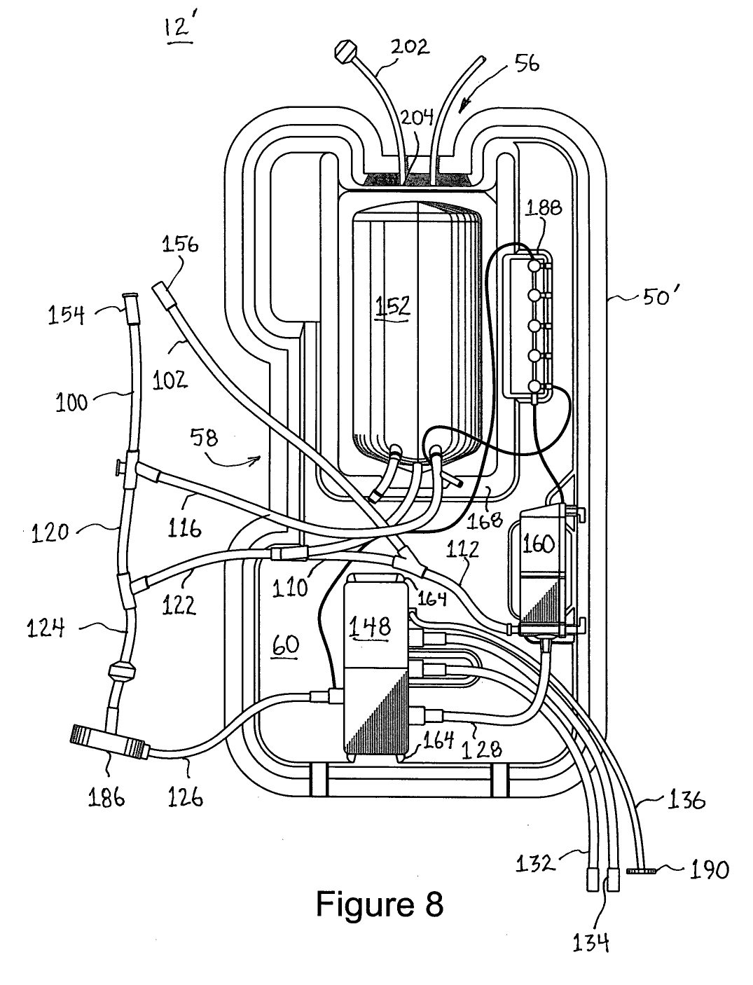

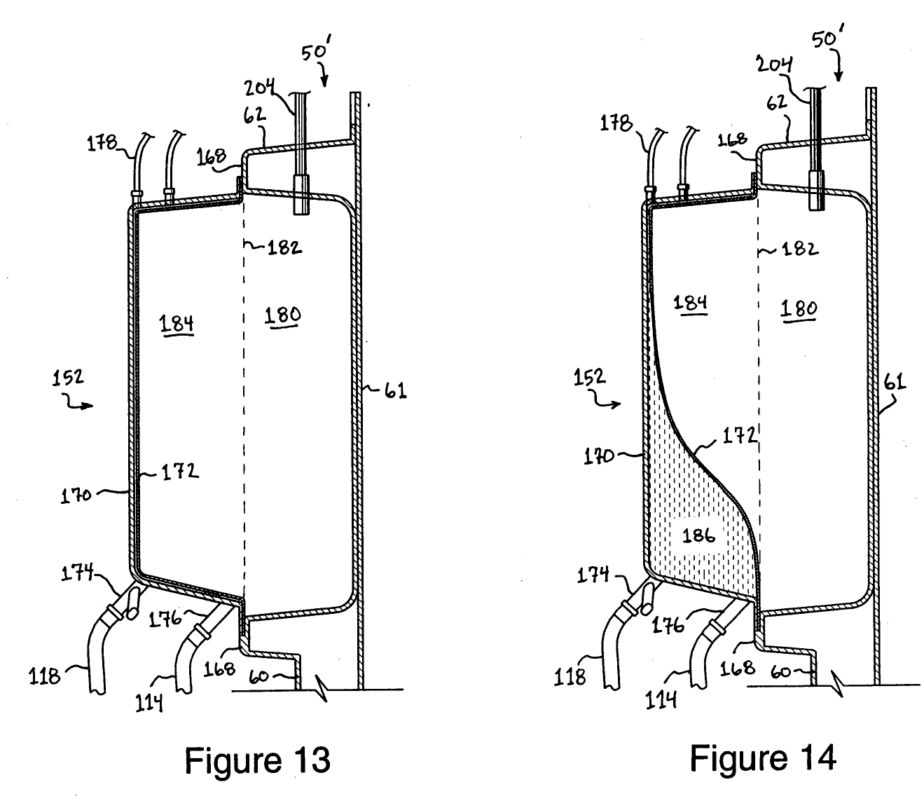

Extracorporeal blood circulation module 12′ comprises all of the components previously described in connection with the embodiment of FIGS. 1-6 and 11-12, and further comprises at least one vacuum line 202 coupled between vacuum source 200 and module 12′. Vacuum line 202 enters the interior of support plane 50′ at a point designated generally with reference numeral 204; as is evident in FIG. 8. The entry of vacuum line 202 into support plane 50′ is depicted in the side cross-sectional views of FIGS. 13 and 14, where it is apparent that vacuum line 202 extends into support plane 50 to be in fluid communication with the vacuum chamber 180/184 defined by rigid wall 170 of reservoir 152 and front surface 60 of support plane 50.

As is apparent from FIGS. 13 and 14, rigid plate 170 of reservoir 152 and front surface 60 of support plane 50 in the area surrounded by raised platform 168 cooperate to define a vacuum-tight chamber within which flexible wall 172 of reservoir 152 is allowed to expand. In particular, flexible wall 172 can be drawn into chamber 180/184 by establishing negative pressure (i.e., a vacuum) in the chamber 180/184.

In the preferred embodiment, reservoir 152 is secured to the front surface 60 of support plane 50 by means of rivets, glue, bonding double-sided tape, or the like, in such a way as to ensure that the chamber defined by indentation 180 and the interior 184 of reservoir 152 (chamber 180/184) comprise an air-tight chamber, such that negative pressure (e.g., a vacuum) created through vacuum port 204 can be created.

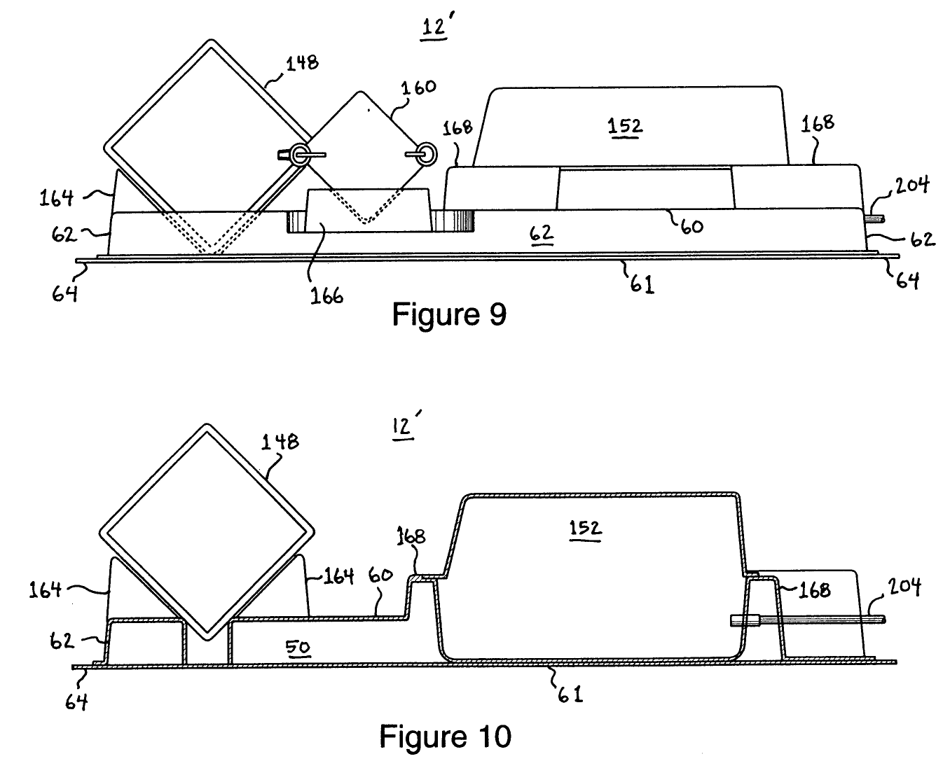

As would be appreciated by those of ordinary skill in the art, the arrangement depicted in FIGS. 7-10 and 13-14 is such that appropriate regulation and control of vacuum source 200 creates the negative pressure within the chamber 180/184, tending to draw membrane 172 into that chamber, thereby exerting suction pressure on venous inlet tubing 118. Through proper control of the vacuum source 200, therefore, the venous drainage flow rate can be precisely controlled.

Although not shown in the Figures, in consideration of the possibility of a breach in membrane 172 allowing blood or other foreign matter to enter the vacuum chamber defined by indentation 180 and reservoir interior 184, it is contemplated that vacuum port 204 may be modified to extend from the top of depression 180 to the bottom of depression 180, such that any foreign fluid entering the vacuum chamber would be immediately withdrawn into vacuum tube 204 and be immediately observable by the perfusionist. Alternatively, vacuum port 204 may be configured to enter the vacuum chamber through the rear surface 61 of support plane 50′, although this alternative has possible disadvantages in terms of packaging considerations.

Those of ordinary skill in the art may recognize that if for some reason pump 186 were to fail while vacuum pressure (negative pressure) is being applied to vacuum chamber 180/184, there is a possibility for retrograde blood flow through arterial filter 160 and oxygenator 148. Arterial blood could be drawn from line 142, through filter 160 and oxygenator 148, the (presumably) failed pump head 186, and into venous reservoir 152. As a precaution against such an undesirable scenario, it is contemplated in one embodiment of the invention to provide a one-way check valve along the length of line 126 (referring to FIG. 6) at the output of pump 186. Such a valve (not shown in the Figures) would block the retrograde flow of blood, and permit blood to flow only in the intended direction through pump 186. Suitable one-way check valves are well-known in the art, and many varieties are commercially-available.

From the foregoing, it will be apparent to those of ordinary skill in the art that a method and apparatus for cardiac bypass procedures has been disclosed which involves the use of a minimalized extracorporeal blood circulation module. Although specific embodiments of the invention have been disclosed, it is to be understood that this has been done solely for the purposes of describing various aspects of the invention, and is not intended to be limiting with respect to the scope of the invention as defined by the claims that follow. It is contemplated that various substitutions, alterations, and/or modifications, including but not limited to those design alternatives specifically mentioned herein, may be made to the disclosed embodiments without departing from the spirit and scope of the invention as defined in the claims.

What is claimed is:

1. An extracorporeal blood circulation module for a cardiac perfusion system, comprising:
a substantially hollow support plane having front, back and side surfaces, said front surface of said support plane having a concave depression formed therein;
at least one blood handling component permanently affixed to said front surface of said support plane;
a venous blood reservoir having at least one flexible wall and at least one rigid wall, said venous blood reservoir being permanently affixed to said front surface of said support plane and covering said concave depression formed in said front surface thereof, said front surface of said support plane and said at least rigid wall of said reservoir thereby cooperating to define a vacuum chamber in fluid contact with said at least one flexible wall.

2. The extracorporeal blood circulation module of claim 1, further comprising:
a vacuum inlet formed in said hollow support plane and adapted to permit a vacuum line to extend into said vacuum chamber defined by said support plane and said at least one rigid wall.

3. The extracorporeal blood circulation module of claim 1, wherein said at least one blood-handling component includes an oxygenator.

4. The extracorporeal blood circulation module of claim 3, wherein said at least one blood-handling component further includes a blood filter.

5. The extracorporeal blood circulation module of claim 4, wherein said front surface of said support plane has structures formed therein adapted to receive said at least one blood handling component and facilitate fixation of said at least one blood handling component to said front surface of said support plane.

6. The extracorporeal blood circulation module of claim 1, further comprising:
a gasket, disposed between said reservoir and said front surface of said support plane.

7. The extracorporeal blood circulation module of claim 1, wherein the priming volume for the module is less than 1000 cubic centimeters.

8. The extracorporeal blood circulation module of claim 1, wherein said support plane is made of thermoformed high-density polystyrene.

9. A support structure for a plurality of blood-handling components in an extracorporeal blood circulation circuit, comprising:
front, back, and side walls arranged in a substantially parallelepiped configuration;
an indentation formed in said front wall of said support structure and adapted to cooperate with a rigid wall of a venous blood reservoir to define a vacuum chamber between said reservoir and said front wall of said support structure.

10. The support structure of claim 9, further comprising:
at least one additional indentation formed in said front wall of said support structure adapted to securely receive one of: a blood oxygenator and a filter.

11.

12. The support structure of claim 9, further comprising:
a vacuum port, formed in said support structure, adapted to receive a vacuum line therein and to permit said vacuum line to extend into said vacuum chamber.

13. The support structure of claim 9, wherein said support structure is made of thermoformed high-density polystyrene.

14. A method of performing a heart bypass operation, comprising:
(a) providing an extracorporeal blood circulation module having a substantially hollow support plane with front, back and side surfaces, said front surface of said support plane having a concave depression formed therein;
(b) providing at least one blood handling component permanently affixed to said front surface of said support plane;
(c) providing a venous blood reservoir having at least one flexible wall and at least one rigid wall, said venous blood reservoir being permanently affixed to said front surface of said support plane and covering said concave depression formed in said front surface thereof, said front surface of said support plane and said at least rigid wall of said reservoir thereby cooperating to define a vacuum chamber in fluid contact with said at least one flexible wall.

15. A method in accordance with claim 14, further comprising:
(d) introducing a vacuum source into said vacuum chamber.

16. The method of claim 14, further comprising:
(d) forming a vacuum inlet in said hollow support plane to permit a vacuum line to extend into said vacuum chamber defined by said support plane and said at least one rigid wall.

17. The method claim 14, wherein said at least one blood-handling component includes an oxygenator.

18. The method of claim 17, wherein said at least one blood-handling component further includes a blood filter.

19. The of claim 16, further comprising:
(e) forming structures on said front surface of said support plane adapted to receive said at least one blood handling component and facilitate fixation of said at least one blood handling component to said front surface of said support plane.

20. The method of claim 14, further comprising:
(d) disposing a gasket between said reservoir and said front surface of said support plane.

21. The method of claim 14, further comprising:
(d) priming said module with less than 1000 cubic centimeters of priming fluid.

22. The method of claim 14, further comprising:
(d) forming said support plane out of of thermoformed high-density polystyrene.

23. A minimized, integrated extracorporeal blood circuit module, comprising:
a support plane for carrying blood-handling components of the module and interconnecting tubing between said blood-handling components;
a soft-shell venous blood reservoir having at least one rigid wall and at least one flexible wall;
a recess formed in a front surface of said support plane;
a vacuum port adapted to permit extension of a vacuum source into said recess;
wherein said reservoir is sealably mounted on said support plane covering said recess, such that said rigid wall of said reservoir and said front surface of said support plane in the area of said recess cooperate to define sealed vacuum chamber in fluid communication with said flexible wall of said reservoir.

24. The blood circuit module of claim 23, further comprising:
a blood oxygenator, rigidly attached to said front surface of said support plane.

25. The blood circuit module of claim 24, further comprising:
a filter, rigidly attached to said front surface of said support plane.

26. The blood circuit module of claim 23, wherein said support plane is made of thermoformed high-density polystyrene.

27. An extracorporeal blood circulation module for a cardiac perfusion system, comprising:
a rigid support plane having a front surface and a concave depression formed in said front surface;
a venous blood reservoir having flexible wall and a rigid wall, said venous blood reservoir being permanently affixed to said front surface of said support plane covering said concave depression;
wherein said rigid wall cooperates with said concave depression to define a vacuum chamber adjacent to said at least one flexible wall.

28. The extracorporeal blood circulation module of claim 27, further comprising:
a vacuum port formed in said support plane adapted to introduce negative pressure in said vacuum chamber.

29. The extracorporeal blood circulation module of claim 28, further comprising:
a blood oxygenator, fixedly mounted on said front surface.

30. The extracorporeal blood circulation module of claim 29, further comprising:
a blood filter, fixedly mounted on said front surface.

31. The extracorporeal blood circulation module of claim 30, further comprising:
interconnective tubing coupled between said venous blood reservoir, said blood oxygenator, and said blood filter.

32. The extracorporeal blood circulation module of claim 27, wherein said support plane is made of thermoformed high-density polystyrene.
